(19) United States
(12) Patent Application Publication
(10) Pub. No.: US 2024/0157100 A1
(43) Pub. Date: May 16, 2024

(54) BALLOON DILATATION CATHETER AND ANGIOPLASTY USING THE SAME

(71) Applicant: THE CATHOLIC UNIVERSITY OF KOREA INDUSTRY-ACADEMIC COOPERATION FOUNDATION, Seoul (KR)

(72) Inventor: Myunhee LEE, Daejeon (KR)

(21) Appl. No.: 18/362,007

(22) Filed: Jul. 31, 2023

(30) Foreign Application Priority Data
Nov. 14, 2022 (KR) 10-2022-0152093

(51) Int. Cl. A61M 25/10; A61M 25/09

(52) U.S. Cl. CPC A61M 25/104; A61M 25/09

(57) ABSTRACT

One embodiment provides a balloon dilatation catheter. The balloon dilation catheter, according to the embodiment, comprises a catheter tube provided to be disposed in a blood vessel, a balloon connected to the catheter tube and dilatated by fluid injection, a guide tube disposed along the catheter tube in the balloon and one or more bypass tubes located inside the balloon.

### CROSS-REFERENCE TO RELATED APPLICATION

The present application claims priority to Korean Patent Application No. 10-2022-0152093, filed on Nov. 14, 2022, the entire contents of which are incorporated herein for all purposes by this reference.

### BACKGROUND OF THE INVENTION

### Field of the Invention

The present disclosure relates to a balloon dilatation catheter and balloon angioplasty using the same, and more particularly, to a balloon dilatation catheter with an improved structure to solve the disadvantages of the balloon dilatation catheter blocking blood flow during angioplasty and not being easy to reuse because it cannot be restored to its original shape after use, and balloon angioplasty using the same.

### Description of the Related Art

In general, a catheter is a tube inserted into a body cavity (pleural cavity, peritoneal cavity), organs (trachea, esophagus, stomach, intestines, bladder, ureters, blood vessels), and the like. Conventionally, a catheter is used to measure the discharge of the inner fluid of organs, but it has been used for purposes such as securing a passage for the injection of a drug solution or insertion of a sensor. These catheters are made of materials such as rubber, plastic, and metal, have various diameters and lengths, and are provided in various ways depending on the area and purpose of use.

In percutaneous transluminal angioplasty (PTA), a balloon dilatation catheter is used. Since the dilatation of the balloon blocks blood vessels and blood flow, there is a risk of various serious complications (e.g., long-term ischemia (myocardial ischemia, etc.), pain, etc.). When deflated, the balloon cannot be restored to its original shape, making it difficult to reuse. Since multiple catheters are used during a procedure, it imposes a significant cost burden on patients and health care institutions.

As Prior Art 1 related to such a balloon dilatation catheter, Korean Patent No. 10-2296255 discloses a “balloon catheter” (hereinafter referred to as Prior Art 1).

Prior art 1 provides a passage capable of maintaining blood flow in aortic blood vessels and is configured to prevent a jumping phenomenon in which the balloon catheter and its associated medical device deviate from the corresponding location due to high blood pressure during the procedure.

Since this prior art is a method of fixing the size of the inner member to maintain blood flow in a blood vessel, it is impossible to shrink the minimum area. In addition, in the case of the outer balloon, a problem occurs when the catheter is reused due to deformation of the shape during contraction.

In addition, as Prior Art 2 related to balloon dilatation catheters, Japanese Patent Application No. 10-2020-177507 discloses “a balloon for a catheter and a balloon catheter” (hereinafter referred to as Prior Art 2).

Prior art 2 proposes a balloon for a catheter that can achieve an original therapeutic purpose without blocking blood flow by the balloon that completely occludes the inside of a blood vessel when the balloon makes close contact with the inside of the blood vessel. A space for blood flow is formed to ensure blood flow between adjacent expansion sections when the balloon for a catheter expands, and a recessed groove communicating with the space for blood flow is provided to ensure blood flow even when the catheter balloon is inflated. Also, a method of completely occluding a blood vessel by pressure control is also possible.

Since the shape of the balloon is deformed in this Prior Art 2 when the balloon is deflated, it is difficult to reuse.

### Documents of Related Art

(Patent Document 1) KR Patent No. 10-2296255, titled “balloon catheter”

(Patent Document 2) Japanese Patent Application No. 10-2020-177507, titled “a balloon for a catheter and a balloon catheter”

### SUMMARY OF THE INVENTION

In order to solve the above problems, an objective of the present disclosure is to provide a balloon dilatation catheter and balloon angioplasty using the same that maintains the flow of blood to the distal part during a procedure by providing a tunnel through which blood flow can pass in a state where blood vessels are blocked during the procedure, so that the stability of the patient can be secured during the procedure and the reuse of the catheter is facilitated after the procedure, and at the same time, cost reduction can be expected.

The technical problems to be solved in the present disclosure are not limited to the above-mentioned technical problems, and any other technical problems not mentioned will be clearly understood from the following description by those skilled in the art.

In order to achieve the above objective, the present disclosure provides a balloon dilatation catheter, comprising a catheter tube provided to be disposed in a blood vessel; a balloon connected to the catheter tube and dilatated by fluid injection; a guide tube disposed along the catheter tube in the balloon; and one or more bypass tubes located inside the balloon extending between the proximal and distal end, wherein blood flow is maintained through the bypass tubes between both sides of the blood vessel blocked by the balloon in a state where the balloon is dilatated.

In one embodiment of the present disclosure, the bypass tube may be supported on both sides of the balloon. It may be made of an elastic material that restores a dilatated shape of the balloon and may include one or more of a spiral, wave, and curved shape.

In one embodiment of the present disclosure, it is appropriate that the bypass tube is spirally arranged surrounding the guide tube.

In one embodiment of the present disclosure, the bypass tube may be connected to a front part of the balloon and a rear part of the balloon, an inlet may be located in the front part of the balloon, and an outlet may be located in the rear part of the balloon.

In one embodiment of the present disclosure, blood flow in the bypass tube may be formed opposite to the entry direction of the balloon.

In one embodiment of the present disclosure, the balloon may contract around the bypass tube when the balloon is deflated and may be supported in contact with the bypass tube.

Furthermore, in order to achieve the above objective, the present disclosure provides balloon angioplasty using the balloon dilatation catheter, wherein a guide wire pre-disposed in the blood vessel is moved through the guide tube so that the balloon is located at a target site of the blood vessel, and the liquid is injected into the balloon through the catheter tube to dilate the balloon, and blood flow is maintained between both sides of the blood vessel through the bypass tube.

### BRIEF DESCRIPTION OF THE DRAWINGS

FIG. 1 is a conceptual view of a procedure in which a balloon dilatation catheter is inserted into a blood vessel according to an embodiment of the present disclosure.

FIG. 2 is a cross-sectional view taken along line A-A′ in FIG. 1.

FIG. 3 is a detailed isolation view of a bypass tube and a guide wire in FIG. 1.

### DETAILED DESCRIPTION OF THE INVENTION

Hereinafter, the present disclosure will be described with reference to the accompanying drawings. The present disclosure, however, may be implemented in different forms, and thus, it is not limited to the embodiments which will be described below. In the drawings, some portions not related to the description will be omitted in order to clearly describe the present disclosure, and similar reference numerals are given to similar components throughout the present disclosure.

Throughout the present disclosure, when a portion is referred to as being “connected” (accessed, contacted, or coupled) to another portion, it may include a case in which the portion is “directly connected” to the other portion as well and a case in which the portion is “indirectly connected” to the other portion with another part interposed therebetween. Also, when a portion is referred to as “including” a component, it may mean that another component is further included and is not to be excluded unless specifically stated otherwise.

The terms used herein are for the purpose of describing only specific embodiments and are not intended to be limited to the present disclosure. Unless the context clearly dictates otherwise, the singular form includes the plural form. In the present disclosure, the terms “comprising,” “having,” or the like are used to specify that a feature, a number, a step, an operation, a component, an element, or a combination thereof described herein exists. They do not preclude the presence or addition of one or more other features, numbers, steps, operations, components, elements, or combinations thereof.

Hereinafter, embodiments of the present disclosure will be described in detail with reference to the accompanying drawings.

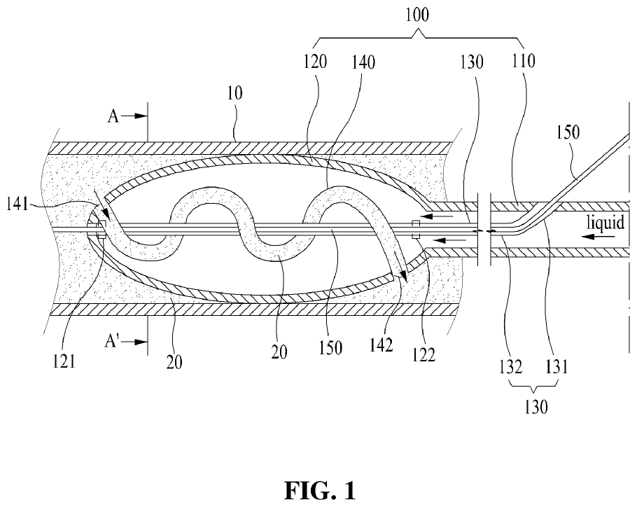

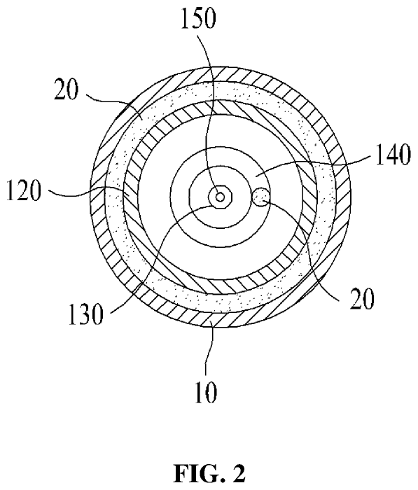

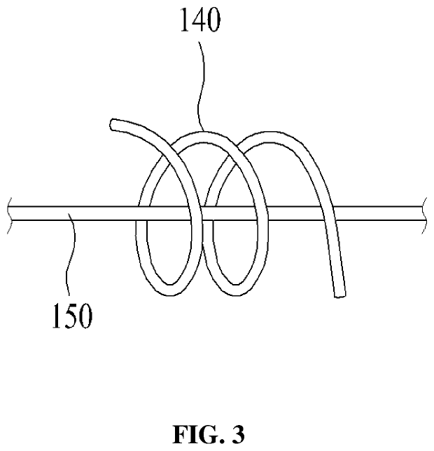

FIG. 1 is a conceptual view of a procedure in which a balloon dilatation catheter is inserted into a blood vessel according to an embodiment of the present disclosure, FIG. 2 is a cross-sectional view taken along line A-A′ in FIG. 1, FIG. 3 is a detailed isolation view of a bypass tube and a guide wire in FIG. 1.

Referring to FIGS. 1 to 3, a balloon dilatation catheter 100, according to an embodiment of the present disclosure, is configured to include a catheter tube 110 provided to be disposed in a blood vessel 10, a balloon 120 connected to the catheter tube 110 and dilatated by fluid injection, a guide tube 130 disposed inside and fixed along the catheter tube 110 in the balloon 120 and used for insertion and removal of a catheter instrument, and one or more bypass tubes 140 located inside the balloon 120 extending between proximal and distal end.

According to the present embodiment, the catheter tube 110 and the balloon 120 connected thereto are located in the appropriate procedure site of the blood vessel 10 along the guide wire 150 inserted into the guide tube 130, and liquid is injected into the balloon 120 through the catheter tube 110 to inflate the balloon 120. Thereafter, in a state in which the dilatation of the balloon 120 blocks the blood vessel 10, the blood flow 20 between both sides of the blood vessel 10 is maintained at a certain level or higher through the bypass tube 140, so that blood flow is secured. Accordingly, it is possible to prevent various serious complications that may occur in the human body due to blockage of blood flow during balloon angioplasty.

The catheter tube 110 is used to inject liquid into the balloon 120 during balloon angioplasty and is provided with a diameter capable of entering and moving into the blood vessel 10. It maintains a certain level of rigidity or elasticity so that the balloon 120 can be pushed along the blood vessel 10, and is made of a material harmless to the human body.

The balloon 120 is connected to the front end of the catheter tube 110 and serves to expand the narrowed blood vessel 10 substantially, and is formed of a membrane material having elasticity and may be dilatated in an elliptical shape to contact the inner wall of the blood vessel 10. The balloon 120 may be made of a soft elastic material, including medical silicone.

On the other hand, the balloon 120 may include a start marker 121 and an end marker 122 provided to correspond to the front and rear parts. These are used to indicate the location of the balloon 120 relative to the guide wire 150 within the blood vessel 10 during the procedure.

The guide tube 130 may include an inclined tube 131 inserted and disposed into the catheter tube 110 and a horizontal tube 132 connected to the inclined tube 131 and connected to the balloon 120. However, the guide tube 130 is not limited thereto and may be formed only of the horizontal tube structure without the inclined tube structure.

The guide tube 130 has a diameter of about ½ to ⅕ of the diameter of the catheter tube 110. The inclined tube 131 of the guide tube 130 has an inlet slightly larger than the horizontal tube 132. It may be formed as a tapered tube that gradually narrows from the inlet toward the horizontal tube 132 to be equal to the size of the horizontal tube 132.

On the other hand, in the process of moving the horizontal tube 132 of the guide tube 130 extending to the balloon 120 through the guide wire 150 first entered into the blood vessel to the destination, the balloon 120 of the catheter 100 enters the blood vessel along the guide wire 150 and is moved to a desired location. In this case, the guide wire 150 is moved to the blood vessel 10 and is in a state in which it is sufficiently extended to the procedure site. To move the balloon 120 along the guide wire 150, the guide wire 150 enters from the front end of the balloon 120, passes through the horizontal tube 132, and then comes out toward the inclined tube 131. Alternatively, in the structure without the inclined tube 131, the guide wire 150 comes out in the horizontal direction as it is.

The start marker 121 and the end marker 122 are located on the guide wire 150. These markers 121 and 122 are used as marks to guide the location of the balloon 120 on the guide wire 150.

The bypass tube 140 is supported on both sides of the balloon 120 and is made of an elastic material that restores the original shape of the balloon 120 and may include one or more of a spiral, wave, and curved shape.

It is appropriate that the bypass tube 140 is spirally arranged surrounding the guide tube 130. Accordingly, in the bypass tube 140, the blood flow 20 spirally rotates more than 360 degrees along the guide tube 130 to pass through the procedure site of the blood vessel 10 blocked by the balloon 120, thereby creating a blood flow above a certain level, which does not cause complications to the human body.

In addition, the bypass tube 140 is connected to the front part of the balloon 120 and the rear part of the balloon 120, an inlet 141 may be located in the front part of the balloon 120, and an outlet 142 may be located in the rear part of the balloon 120.

In the bypass tube 140, the blood flow 20 flows into the inlet 141, rotates in a spiral direction, and then flows out into the outlet 142, thereby passing through the bypass tube 140. Therefore, a blood flow opposite to the entry direction of the balloon 120 is formed, but is not necessarily limited thereto, and the blood flow 20 may be formed opposite to the above flow between the inlet 141 and the outlet 142.

On the other hand, the catheter 100 for balloon angioplasty may be discharged to the outside only when the balloon 120 is deflated by discharging the liquid injected into the balloon 120 to the outside after the procedure of the blood vessel 10. In this case, the balloon 120 contracts around the bypass tube 140 when deflated and is supported in contact with the bypass tube 140. That is, after the liquid is discharged, the balloon 120 is not flatly contracted but maintains its smallest shape by being supported in contact with the spiral outer circumferential surface of the spiral bypass tube 140. This is because the balloon 120 is contracted around the bypass tube 140 (compared to a conventional balloon catheter that is contracted flat) to minimize the area of the balloon after contraction so that it is easy to reuse the catheter later and to minimize damage to blood vessels during the repeated procedures.

Hereinafter, balloon angioplasty, in which blood flow is maintained according to an embodiment of the present disclosure will be described with reference to FIGS. 1 to 3.

Referring to FIGS. 1 to 3, balloon angioplasty, in which blood flow is maintained according to an embodiment of the present disclosure, uses a balloon angioplasty catheter 100 in which blood flow is maintained. In balloon angioplasty, the balloon 120 is moved along the guide wire 150 pre-arranged in the blood vessel 10 to be located at the target site of the blood vessel 10, and liquid is injected into the balloon 120 through the catheter tube 110 so that the balloon 120 is dilatated. Thus, blood flow 20 is maintained between both sides of the blood vessel 10 blocked by the balloon 120 through the bypass tube 140 to prevent complications that may occur in the human body due to blood vessel blockage during angioplasty.

Existing inventions do not fulfill the purpose of maintaining blood flow (e.g., fixing the location of the balloon in the blood vessel), or the empty space inside the balloon is not uniform and is biased in one direction within the catheter, so that there is a fatal disadvantage that can reduce the effectiveness of angioplasty during balloon dilatation. For example, in an existing balloon dilatation catheter with an empty space inside the balloon, pressure is not evenly distributed during balloon angioplasty due to the influence of lesions such as blood clots/plaques in the blood vessels, which reduces the effect of balloon angioplasty, or there is a possibility that the space inside the balloon will be compressed by external pressure.

In the above-described present disclosure, which has solved the above problems by providing a bypass tube that rotates 360 degrees around the central axis inside the balloon connected to the catheter tube as a spiral blood flow tunnel, blood flow is maintained during balloon dilatation, resulting in preventing complications due to blood flow interruption.

In addition, there is a tunnel in the balloon of the catheter that rotates 360 degrees around the central axis, which evenly distributes the external pressure caused by intravascular lesions (thrombosis, calcific plaque, atherosclerotic plaque, etc.), thereby minimizing the decrease in the effect of angioplasty caused by the inner tunnel during balloon dilatation.

In addition, in the present disclosure, since there is a tunnel rotating 360 degrees around the central axis inside the balloon catheter, which can minimize the area when the balloon is deflated after angioplasty, it is easy to reuse the catheter later. In addition to the cost-saving effect of avoiding additional balloon catheters, especially in complex lesion procedures (for example, when multiple balloon catheters are used simultaneously in branch vascular procedures), the use of additional tools (e.g., large-diameter guiding catheters, etc.) can be reduced. Therefore, it also provides an advantage that the procedure can be performed more quickly and safely. The method of using the catheter of the present disclosure is the same as the conventional catheter for balloon angioplasty. Therefore, all other tools or devices (e.g., guide wire, guiding catheter) that are used during the procedure can be used in the same manner. Also, the number, size (diameter), angle, and spiral rotation of the bypass tube in the balloon can be modified according to the purpose of the procedure, and the bypass tube can be applied to balloon catheters of various sizes, shapes, and materials. For example, the bypass tube can be applied to catheters for balloon angioplasty of all blood vessels, such as coronary artery, peripheral artery, aorta, cerebral artery, and vein. Meanwhile, the catheter of the present disclosure can be applied to other types of catheters (e.g., stents, drug-eluting balloons, etc.) in which balloon dilatation catheters are used in the same manner.

Due to the effect of the present disclosure according to the configuration as described above, it can provide a catheter for balloon angioplasty that maintains blood flow to the distal part during the procedure, thereby securing the stability of the patient during the procedure and facilitating the reuse of the catheter after the procedure, thereby reducing the cost at the same time.

It should be understood that the effects of the present disclosure are not limited to the above-described effects and include all effects deduced from the detailed description of the present disclosure or the configuration thereof defined by the appended claims.

The above-described description of the present disclosure is intended only for illustrative purposes, and it can be easily understood that other concrete forms can be devised by those skilled in the art without changing or modifying the technical spirit or essential characteristics of the present disclosure. Therefore, it should be understood that the above-described embodiments are not restrictive but illustrative in all aspects. For example, each component described as a single form may be distributed and implemented, and similarly, components described as being distributed may also be implemented in a combined form.

The appended claims define the scope of the present disclosure, and all alternations or modifications derived from the meaning and scope of the claims and their equivalents should be construed as being included within the scope of the present disclosure.

### DESCRIPTION OF REFERENCE NUMERALS

10: blood vessel
20: blood flow
100: catheter
110: catheter tube
120: balloon
121: start marker
122: end marker
130: guide tube
131: inclined tube
132: horizontal tube
140: bypass tube
141: inlet
142: outlet
150: guide wire

1. A balloon dilatation catheter, comprising:
a catheter tube configured to be disposed in a blood vessel;
a balloon connected to the catheter tube and configured to be dilatated by fluid injection;
a guide tube disposed along the catheter tube in the balloon; and
at least one bypass tube disposed through the balloon,
wherein blood flow is maintained through the at least one bypass tube between both sides of the blood vessel blocked by the balloon in a state where the balloon is dilatated and the blood vessel is blocked.

2. The balloon dilatation catheter of claim 1, wherein the at least one bypass tube is supported on both sides of the balloon and is made of an elastic material that restores a dilatated shape of the balloon, and has a spiral shape, a wave shape, or a curved shape.

3. The balloon dilatation catheter of claim 2, wherein the at least one bypass tube is spirally arranged surrounding the guide tube.

4. The balloon dilatation catheter of claim 2, wherein the at least one bypass tube is connected to a front part of the balloon and a rear part of the balloon, an inlet of the at least one bypass tube is located in the front part of the balloon, and an outlet of the at least one bypass tube is located in the rear part of the balloon.

5. The balloon dilatation catheter of claim 4, wherein blood flow in the at least one bypass tube is directed opposite to an entry direction of the balloon.

6. The balloon dilatation catheter of claim 3, wherein the balloon contracts around the at least one bypass tube when the balloon is contracted, and is supported in contact with the at least one bypass tube.

7. A balloon dilation angioplasty using the balloon dilatation catheter of claim 1, wherein a guide wire pre-disposed in the blood vessel is moved through the guide tube so that the balloon is located at a procedure site of the blood vessel, and liquid is injected into the balloon through the catheter tube to dilate the balloon, and the blood flow is maintained between the both sides of the blood vessel blocked by the balloon through the at least one bypass tube.
